(19) United States
(12) Patent Application Publication
(10) Pub. No.: US 2018/0305743 A1
(43) Pub. Date: Oct. 25, 2018

(54) METHOD OF DETECTING AND IDENTIFYING MICROBIAL SOURCES OF CONTAMINATION IN WATER SYSTEMS

(71) Applicant: MICROBE DETECTIVES LLC, Downers Grove, IL (US)

(72) Inventor: Trevor Wayne GHYLIN, Wauwatosa, WI (US)

(21) Appl. No.: 15/961,776

(22) Filed: Apr. 24, 2018

Related U.S. Application Data
(60) Provisional application No. 62/488,918, filed on Apr. 24, 2017.
(60) Provisional application No. 62/488,913, filed on Apr. 24, 2017.
(60) Provisional application No. 62/501,857, filed on May 5, 2017.

(51) Int. Cl. C12Q 1/689; G01N 1/18; G01N 33/18

(52) U.S. Cl. CPC C12Q 1/689; G01N 1/18; G01N 2001/1031; C12Q 2600/16; G01N 33/1806

(57) ABSTRACT

The present invention relates to a method of detecting contaminants of microbial origin in a water distribution system and, more particularly, to a method of identifying the microbes contaminating the system, by manipulation of the DNA of the microbes. The microbes are collected, and a portion of the DNA sequence of a particular gene that is found in microbes is amplified. The portion of the DNA sequence that is amplified contains a highly variable region and the identity of the microbe can be determined by that DNA sequence. Each amplified DNA sequences is matched against the known sequences of corresponding regions in the DNA sequences of various microbes. Thus, the spectrum of microbial contaminants, and their relative quantities, can be identified in the liquid collected from different locations of the water distribution system.

### CROSS-REFERENCE TO RELATED APPLICATIONS

This application claims priority to and benefit of application Ser. No. 62/488,913 filed Apr. 24, 2017; Ser. No. 62/488,918 filed Apr. 24, 2017; and Ser. No. 62/501,857, filed May 5, 2017, which are hereby incorporated herein by reference in their entirety.

### FIELD OF THE INVENTION

The present invention relates to a method of detecting contamination of a water system and, more particularly, to a method of detecting contamination using DNA identification.

### BACKGROUND OF THE INVENTION

The present invention relates to a method of detecting contamination of a water system and, more particularly, to a method of detecting contamination using DNA identification. More particularly, the present relates to using microbial DNA sequencing to identify the presence and sources of contamination in a water system including both naturally occurring water systems and man-made water distribution systems.

Detecting and locating problems (for example, backflow, cross-connections, contamination, intrusion) in drinking water distribution systems is often impossible because the contamination is usually a small amount and chlorine that is often added to the water prevents contaminant bacteria from growing in typical water quality tests. Routine inspection is very labor intensive and will not detect problems for long periods of time. Water quality monitoring based on culturing may not detect problems as contaminant microbes are suppressed by chlorine and may not grow in culture.

Water systems can include naturally occurring water systems such as streams, rivers, lakes, beaches, and other bodies of water in which it may be desirable to test for microbial contamination. Water systems can also include man-made water systems such as drinking or industrial water distribution systems, irrigation systems, retaining ponds, canals, channels, and other bodies of water where it may be desirable to test for microbial contamination. Microbial contaminants can enter such systems at a variety of sites or points and in variety of ways. For example, contamination can enter the system from agricultural run-off, sewage systems, system entry ports, backflow, cross-connections, and intrusions. It is often difficult even to detect microbes in these systems. The microbes are too small to detect with the naked eye and are often present in quantities too small for detection by conventional methods. For example, microbes may often be too small and too dispersed to detect by histological methods that depend on microscopy.

Previously known strategies employed in monitoring and investigating water systems such as distribution systems can include regularly-scheduled visual and physical inspection of inlets and outlets. Other strategies that may be used in preventing such contamination may include backflow preventers in buildings. Such monitoring, investigation, inspection, and remediation can be very labor-intensive and is often performed annually or even less frequently.

Routine visual inspection of water distribution systems is very labor intensive and sometimes will not detect problems for long periods of time, or until microbes are present in large numbers. Even when microbes are detected by known methods, it can be difficult or impossible to determine the types of microbes that are present and to identify the source of the contamination. This difficulty in turn can make it difficult or impossible to formulate a plan to cure or remediate the contamination.

Many water distribution systems contain features that interfere with certain microbial detection methods. For example, microbes can be grown in culture until they multiply into sufficient numbers for detection and analysis, though this method requires sufficient time for several generations of microbial reproduction. However, water distribution systems can include chlorine (or other additives or preservatives) that can inhibit certain bacteria or microbes from growing in these types of water quality tests. Water quality monitoring based on the culturing of the microbes may not detect contaminant microbes in these conditions. Alternatively, significant amounts of contamination can go undetected because the chlorine (or other additive) in the water suppresses the bacteria's ability to grow in culture.

Even where conventional tests can detect the presence of microbes, those microbes can be present in quantities too small to identify the exact kinds of microbes that are present. Further, microbes can be present in complex mixtures. In mixed samples, microbes present in greater abundance can mask the presence of microbes present in lesser quantities. It can be particularly difficult to identify the microbes that are present in heterogeneous combinations.

Also, many water quality tests are limited to identifying the presence of microbial contaminants, without identifying the contaminating microbes themselves, or are limited to identifying a narrow spectrum of microbes.

As can be seen, there exists a need for an improved method of detecting microbial contamination in water or liquid distribution systems, particularly when the microbes are dead, unable to reproduce, or present in very small quantities when the samples are analyzed. There is a further need for determining the identity of the microbes present in those samples, so that appropriate responses can be made to fix or remove the contamination in the water transportation system.

### SUMMARY OF THE INVENTION

The present invention in one embodiment relates generally to a method of detecting and identifying multiple microbes that are present as contaminants in a water distribution system, or determining if certain microbes are absent from the system. One aspect of the invention relates to a method of collecting samples from the water distribution system and analyzing the DNA of the microbial contaminants present in the sample. This method targets the DNA of a gene that is commonly found in most microbes, but that commonly-shared gene also contains a hypervariable region with a DNA sequence unique to each species, genus, family, order, class, or phylum of microbe.

The DNA of each contaminating microbe is collected and then amplified to sufficient quantities to determine the unique sequence of the hypervariable region of each microbe. Determining the unique sequence of the hypervariable region thus provides the identity of each contaminating microbe. This allows the identification of multiple different microbes in the same sample and can provide relative quantities of each microbe or family of microbes.

An embodiment of the method of this invention can be used to test for microbial contamination in naturally occurring bodies of water, such as on a beach, lake, river, or other. In an embodiment of the invention, identifying the specific microbes in the contamination can facilitate identifying the source of the contamination, such as from agricultural run-off, human generated waste, or other.

An embodiment of the method of this invention can be used to test for microbial contamination in a water distribution system at the point of delivery from the source of supply to the user. Water delivery systems may typically be tested at the origin, such as a water treatment plant, but are less often tested at the point of delivery, which may be many miles from the source of origin at the hook-up for a building, residence, industry, hospital, or other. In an embodiment of the invention, testing for contamination and identifying by their DNA the microbes contaminating the system can facilitate identifying the source of contamination.

An aspect of an embodiment of the invention includes comparing the microbial DNA determined from the hypervariable region with known databases of microbial DNA to identify the particular microbes contaminating the water system, which can facilitate identifying the source of contamination and providing remediation and/or cure for the contamination.

Another aspect of this invention relates to a kit for collecting samples from water systems including naturally occurring water systems, artificial water systems, and water distribution systems, said kit being used to obtain, collect, store, and/or transport a sample for further DNA analysis of any contaminating microbes.

### BRIEF DESCRIPTION OF THE SEQUENCE LISTING

The invention be more fully understood from the following detailed description and the accompanying Sequence Listing, which form a part of this application.

The sequence descriptions summarize the Sequence Listing attached hereto. The Sequence Listing contains standard symbols and format used for nucleotide sequence data comply with the rules set forth in 37 C.F.R. § 1.822.

### BRIEF DESCRIPTION OF THE DRAWINGS

The invention can best be understood by reference to the following description taken in conjunction with the accompanying drawings, which are not to scale.

FIG. 1 provides a chart of the steps for detecting and identifying microbial contaminants in a distribution system.

FIG. 2 provides a flow chart of the steps for detecting and identifying microbial contaminants in a distribution system, and shows an exemplary kit including the components for collecting samples for microbial analysis, the kit containing a syringe with filter attachment, sterile filter, desiccant card, processing form, and set of instructions.

FIGS. 3A-3B and 4A-4I show the identification of microbes in a sample.

FIG. 3A shows a representation of a portion of a double-stranded DNA helix.

FIG. 3B shows a representation of the complementary deoxyribosenucleotide sequences of a portion of double-stranded DNA.

FIG. 4A shows the identity and information about bacteria found in a sample.

FIG. 4B shows the identity and information about coliforms found in a sample.

FIG. 4C shows the identity and information about non-coliforms that can trigger coliform test and fecal indicators found in a sample.

FIG. 4D shows the identity and information about contamination indicators found in a sample.

FIG. 4E shows the identity and information about freshwater marine bacteria and nitrogen fixation found in a sample.

FIG. 4F shows the identity and information about carbon fixation found in a sample.

FIG. 4G shows the identity and information about ammonia oxidation and nitrate oxidation found in a sample.

FIG. 4H shows the identity and information about iron oxidation and sulfate reduction found in a sample.

FIG. 4I shows the identity of and information about sulfur oxidation, methane oxidation, and biofilm slime formers found in a sample.

FIG. 5A shows a flow diagram of a water distribution system.

FIG. 5B depicts sources of contamination in a water system.

FIG. 6 shows a kit including the components for collecting samples for microbial analysis.

### DETAILED DESCRIPTION OF THE INVENTION

While the present invention is susceptible of embodiments of various forms, there is shown in the drawings, and will hereinafter be described some exemplary and non-limiting embodiments, with the understanding that the present disclosure is to be considered an exemplification of the invention. It is not intended to limit the invention to the specific embodiments listed. The description is not to be taken in a limiting sense, but is made merely for the purpose of illustrating the general principles of the invention. The scope of the invention is set forth in the appended claims.

Moreover, while an embodiment of the present invention is described in terms of microbial contaminants in a water system, it will be appreciated that microbial contaminants can occur in other liquids and that it is within the scope of an embodiment of the present invention to identify microbial contamination in liquid systems where the liquid is water and/or where the liquid is a liquid other than water.

A microbe is a microorganism, especially a bacterium. Such a bacterium can produce, for example, disease or fermentation. These microorganisms can exist as single cells or colonies of cells. Examples of microbes include Salmonella, E. coli, Enterococcus, cyanobacteria, human-associated bacteria, avian associated bacteria, and others.

DNA, or deoxyribonucleic acid, is a self-replicating material present in most living organisms, including microbes. It provides the genetic instructions for the growth, functioning, and reproduction of living organisms, including microbes.

PCR, or polymerase chain reaction, refers to a technique used in molecular biology to amplify a single copy or a few copies of a segment of DNA in amounts up to several orders of magnitude.

QIIME is an open-source bioinformatics tool for performing analysis of raw microbial DNA sequences, to determine the identity of the microbe from which the DNA was obtained.

RNA, or ribonucleic acid, is a material that is essential in various biological roles in coding, decoding, regulation, and expression of genes.

Collecting samples of water or other liquid as discussed herein may include but is not limited to physically retrieving the sample from a water system, or receipt of a previously received sample at a laboratory or other facility for other and further analysis.

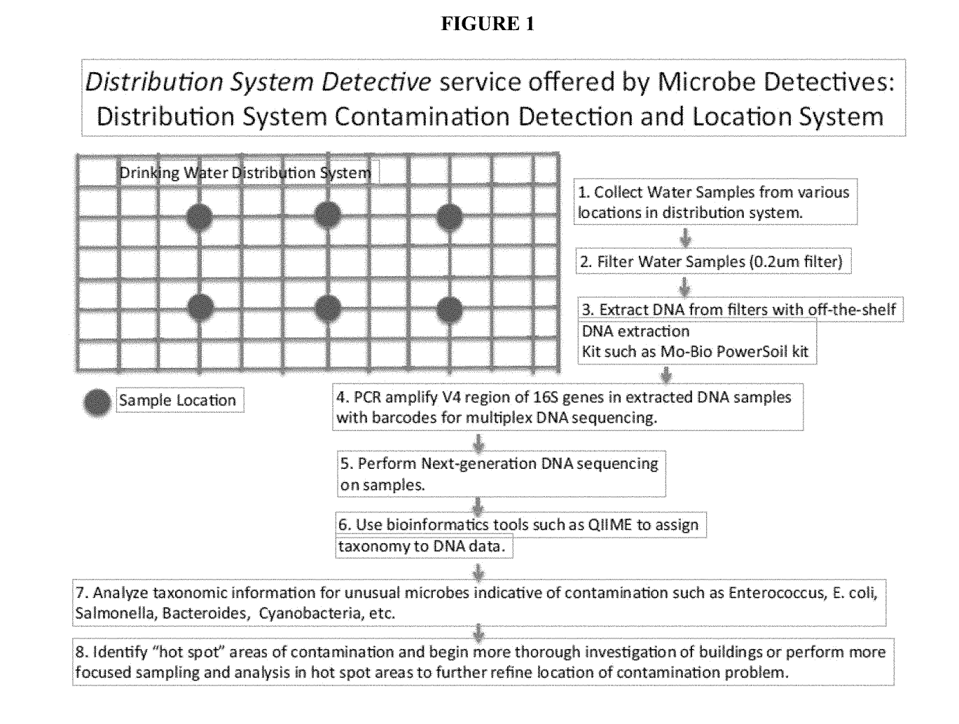

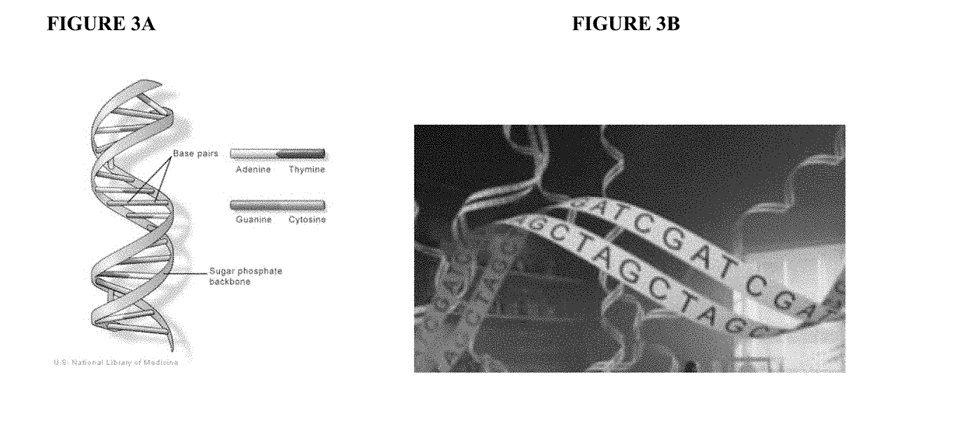

As shown in FIGS. 1-6, broadly, an embodiment of the present invention provides a method of detecting microbial contamination of water systems including water distribution systems, comprising the steps of: collecting water samples from at least one source such as, for example, a water distribution system outlet or other location in the system; filtering the water samples to concentrate the microbes; extracting the DNA of the collected microbes from the filters; amplifying a unique portion of the microbial DNA; determining the sequence the extracted and amplified DNA; and using those DNA sequences to identify the microbe or microbes that were present in the collected water sample by comparing to tables, databases, and the like of known microbial DNA.

One aspect of an embodiment of the present invention relates to a DNA-based microbial analysis service to detect different sources of microbial contamination in a water distribution system. Microbial contamination can be introduced at inlets, outlets, and at connections and cross-connections in the water system. Contaminating microbes can be introduced through normal water flow through the system, or through backflow, sites where water routes connect, contamination of the external surface, intrusion, and other contamination points.

An embodiment of present invention can detect minute amounts of contaminating microbes. An embodiment of the present invention can detect and identify microbes even if the microbes are dead or unable to proliferate normally, such that they could not be grown in a conventional culture, for example. An embodiment of the present invention can also indicate the source of contamination, because many microbes are associated with specific sources. For example, salmonella and/or enterococcus avium are indicative of contamination from birds, especially chickens. Other microbes are associated with particular plants or animals, particular industries, or particular agricultural or industrial processes. Identifying the particular contaminants associated with the microbial contamination can facilitate identification of the source of the contamination.

An embodiment of the present invention can detect microbes that are dead or that otherwise fail to grow using cell culture techniques. An embodiment of the invention is able to detect such microbes because the detection and identification techniques are based on the presence of DNA, instead of requiring viable cells that are capable of reproduction. An embodiment of the present invention can thus identify microbial contaminants even if the cells are dead or degraded, as long as the cells' DNA is sufficiently intact. An embodiment of the present invention can include the sensitive detection and identification of microbes, regardless of whether the cells are viable or not viable when the samples are collected and when the analysis is performed. An embodiment of the present invention can therefore detect contamination that is likely undetectable with traditional culture based water quality tests.

Further, the detection method can be tailored to investigate specific areas for inspection, such as entry and exit points in the water delivery system, rather than having to inspect every building or every unit across the entire system.

The present invention includes water sampling and analysis of microbial data to determine if there is contamination and if so, to facilitate determining and identifying the likely source or sources of the contamination. The other steps of the method can be modified to incorporate any DNA sequencing methodology currently available, or to incorporate future methods of DNA sequencing.

Persons of ordinary skill in the art will appreciate that there are known many commonly-used methods for sequencing the DNA of collected samples, although the application of such technology to identifying contaminants in water systems is novel. For example, an embodiment of the present invention can be practiced by traditional polymerase chain reaction (PCR) and standard DNA sequencing techniques, such as 16S ribosomal deoxyribonucleic acid (rDNA)-based denaturing gradient gel electrophoresis (DGGE) and terminal restriction fragment length polymorphism (T-RFLP) molecular fingerprinting methods, cloning, and more. Embodiments of present invention are intended to encompass all DNA-based technologies routinely used to detect and identify microbes, based on the DNA sequence of those microbes, for the purpose of detecting microbial-sourced contamination problems in distribution systems, and equivalent processes including later developed processes that encompass the functionality of known existing processes for this step in an embodiment of the present invention.

Referring now to FIG. 1, an embodiment of the present invention can include the following steps:

1. Collect water samples from one or more water sample location locations in a water system such as a water distribution system. For example, in a man-made water distribution system samples can be collected at any tap or any site in the water system.
2. Filter water samples onto filters, preferably filters with 0.45 um pores, or more preferably 0.22 um pores, to concentrate microbes onto the filter. The pores should be small enough to retain a standard-sized microbe, but large enough to allow fluid to pass.
3. Extract the DNA from the collected microbes with a DNA extraction kit, or using standard DNA extraction methods. Examples of known suitable DNA extraction kits include but are not limited to DNEasy PowerSoil kit from Qiagen.
4. Perform PCR amplification on the extracted DNA samples to amplify a hypervariable region in a gene commonly found in microbes such as, for example, region V3, V4, V5, and/or V6 of the bacterial 16S ribosomal RNA (rRNA) gene.
5. Perform DNA sequencing on the PCR-amplified DNA samples.
6. Use bioinformatics tools such as, for example, QIIME, an open-source bioinformatics pipeline for performing microbiome analysis from raw DNA sequencing data, and Mothur, another open source software package for bioinformatics data processing, particularly in the analysis of DNA from uncultured microbes, to assign taxonomy and identify microbes in samples.
7. Analyze data obtained from bioinformatics tools for the presence of microbes not typically found in such a water system or such a water distribution system. Examples of such microbes indicative of contamination include but are not limited to. Salmonella, E. coli, Enterococcus, cyanobacteria, human-associated bacteria, and others.
8. Perform more focused sampling efforts based on locations and types of microbes revealed from previous analyses if needed, or begin investigation near sites testing positive for microbial infection.

In greater detail, liquid can be obtained from one or more sites in a natural or artificial water system such as, for example, a water distribution system, and at one or more points in time, as desired. Depending on the amount of liquid available and the microbial load, typical samples can range in volume from a few microliters to a few milliliters to several liters.

In an embodiment of the invention the samples are preferably concentrated before subsequent DNA analysis. For example, the microbes can be concentrated by being collected onto a filter or membrane. In an embodiment employing such filtration for such a step of concentrating microbes, the microbes can preferably be collected on a filter with a pore size smaller than a typical microbe. For example, and not by way of limitation, such a filter can have pores of 2 um or smaller, or preserably 0.45 um or smaller, or more preferably pores of 0.22 um or smaller. In an aspect of an embodiment of the invention the liquid can be directly applied to such filters, or applied via reverse osmotic pressure.

In an embodiment of the invention, after the microbes have been applied to the filters in a step of concentrating the microbes, the DNA of those microbes can be extracted from the filter with standard DNA extraction techniques or technologies. For example, the DNEasy PowerSoil Kit (Qiagen) can be used, according to the manufacturer's instructions, to isolate high quality DNA from such samples in a short period of time such as, for example, in about 30 minutes. A person of ordinary skill in the art will appreciate that a number of kits are commercially available that can be used to extract DNA from a solid substrate or platform.

After the microbial DNA has been extracted from the filter (or other DNA collection and/or concentration platform), the microbial DNA can be amplified or multiplied with molecular biology techniques known to persons of ordinary skill in the art and commercially-available products according to the manufacturers' instructions, such as techniques, products, and or processes, described in, for example, T. Maniatis, E. F. Fritsch & J. Sambrook, Molecular Cloning: A Laboratory Manual (Cold Springs Harbor Laboratory 1982).

Microbes or bacteria can contain certain genes in common. For example, microbes commonly contain a 16S rRNA gene. However, that gene contains highly variable regions encoded by sequences of DNA unique to each species or family of microbes. This variation in the DNA sequence of the gene is widely used to identify the species of an individual microbe. The sequence of the 16S rRNA gene and its hypervariable regions are known for many organisms to persons of ordinary skill in the art, and persons of ordinary skill in the art will appreciate that those sequences are identified, described, and indexed on known databases such as, for example, Greengenes and the Ribosomal Database Project.

The prokaryotic 16S ribosomal RNA gene (16S rRNA) contains at least nine variable regions. The variable regions can be used to identify the genus or species of an individual microbe. For example, variable region V4 of the 16S rRNA gene can be amplified using a system, process, or method such as, for example, the MiSeq System (Illumina), and using primers 515F and 806R can be used to amplify the V4 region of the microbial 16s RNA gene. In most species, the fourth hypervariable region in the 16S rRNA gene (which those of skill in the art will understand can be referred to as “the v4 region”) can be analyzed to identify that microbial species.

Examples of such primer sequences include the following:

For primer 515F (SEQ ID: 1), the sequence is: GTGYCAGCMGCCGCGGTAA.

For primer 806RB (SEQ ID: 2), the sequence is: GGACTACNVGGGTWTCTAAT.

Such primers can be used in standard PCR conditions to amplify the variable region V4 of the 16S rRNA gene. Other primers are commercially available for amplifying the V4 region of the 16S rRNA gene. Other primers are routinely used to amplify other hypervariable regions of the 16S rRNA gene. Examples of such regions include but are not necessarily limited to V3, V4, V5, and/or V6 of the bacterial 16S rRNA gene.

In an embodiment of the invention, the amplified microbial DNA, such as amplified regions of the V4 region of the 16S rRNA gene can then be sequenced by commonly-known molecular biology techniques and commercially-available products according to the manufacturers' instructions.

The 16S rRNA gene is a widely used gene marker for genus and species identification and taxonomic significance in bacteria and archaea. The estimated substitution rate for hypervariable regions is thousands of times higher than for highly conserved regions; the genetic differences of these hypervariable regions provide abundant taxonomic information about microbes. Therefore, the 16S gene amplicons obtained from PCR or other molecular biology techniques can be used to make taxonomic identifications based upon bioinformatics alignments of genetic sequences.

18S rRNA is commonly used for phylogenetic analyses in fungi, and it has more hypervariable domains than 16S rRNA. Also, the ITS (internal Transcribed Spacer) region (which includes 5.8S), is deleted in the posttranscriptional process of nuclear rRNA cistron, and is commonly regarded as a universal fungi barcode marker. The ITS region is routinely used for the identification of a broad range of fungi. Compared to 18S, the ITS region has greater variability and can be more suitable as a genetic marker for measuring intraspecific genetic diversity in fungi.

Just as microbes can be identified through DNA sequencing of the 16S rRNA in microbes, so too can fungi present in the wastewater samples be detected and identified by DNA sequencing of the 18S rRNA or ITS regions.

In an embodiment of the invention, after the DNA sequences have been ascertained those sequences can be analyzed to identify their microbial source. The sequences can be compared to the sequences of microbes that are stored on various bioinformatics platforms. For example, QIIME is an open-source bioinformatics tool for analyzing microbiome DNA sequencing data. Also, Mothur is another open source software tool for analyzing the DNA from uncultured microbes. Both tools allow the identification and classification of microbes found in samples.

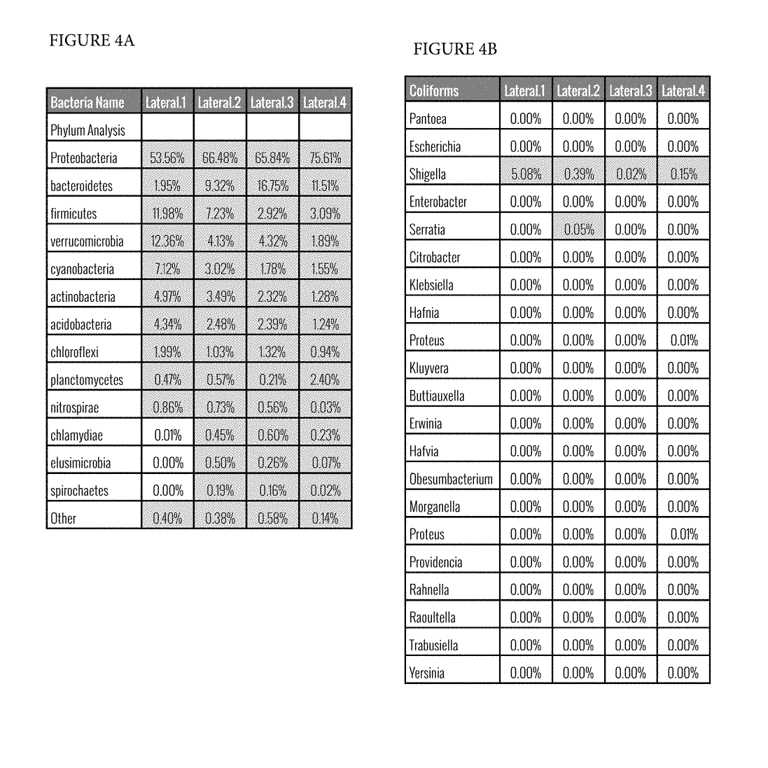

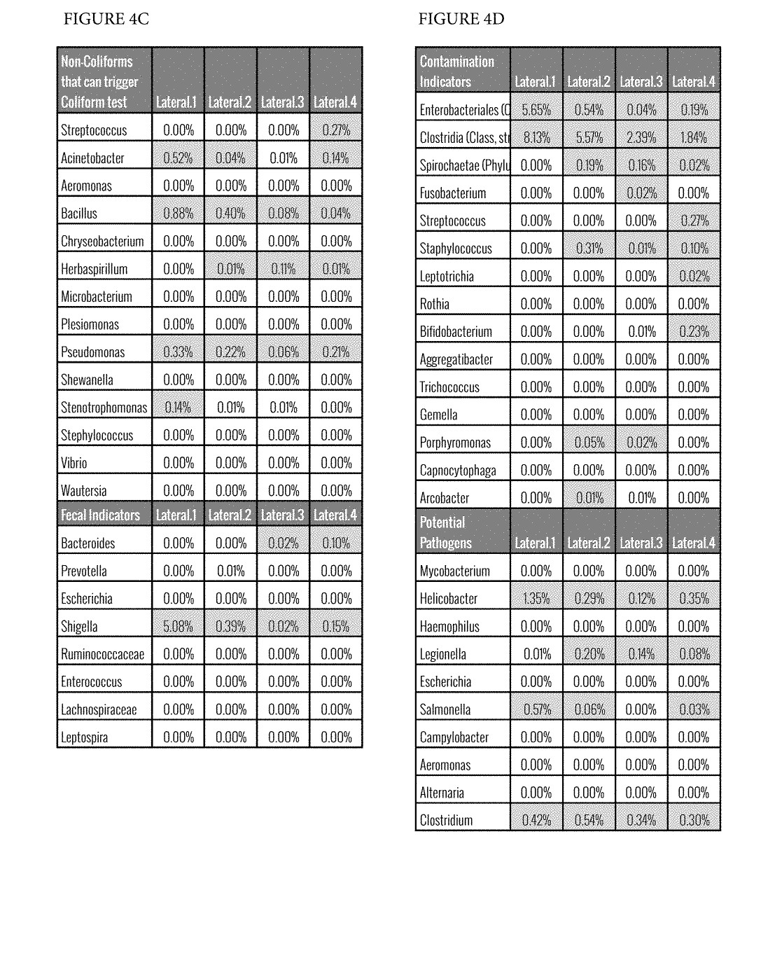

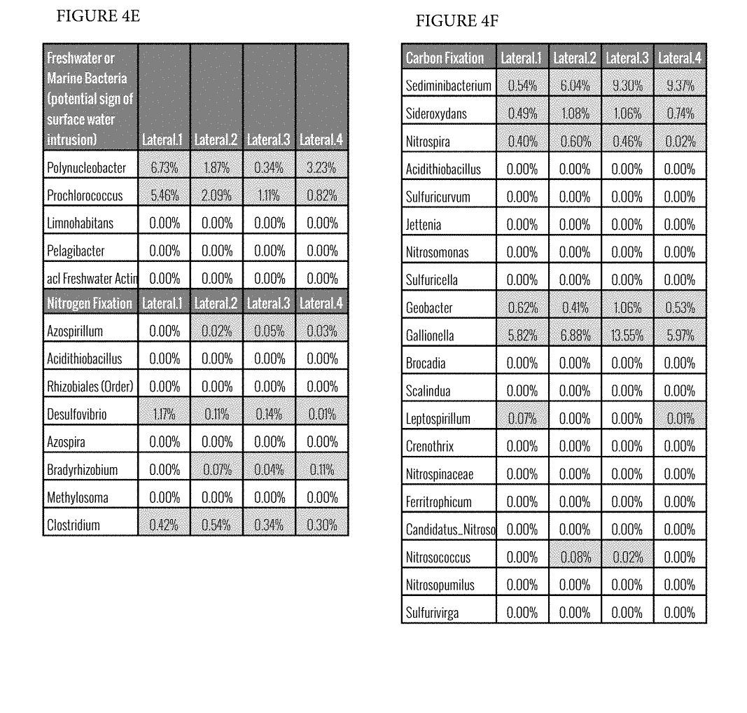

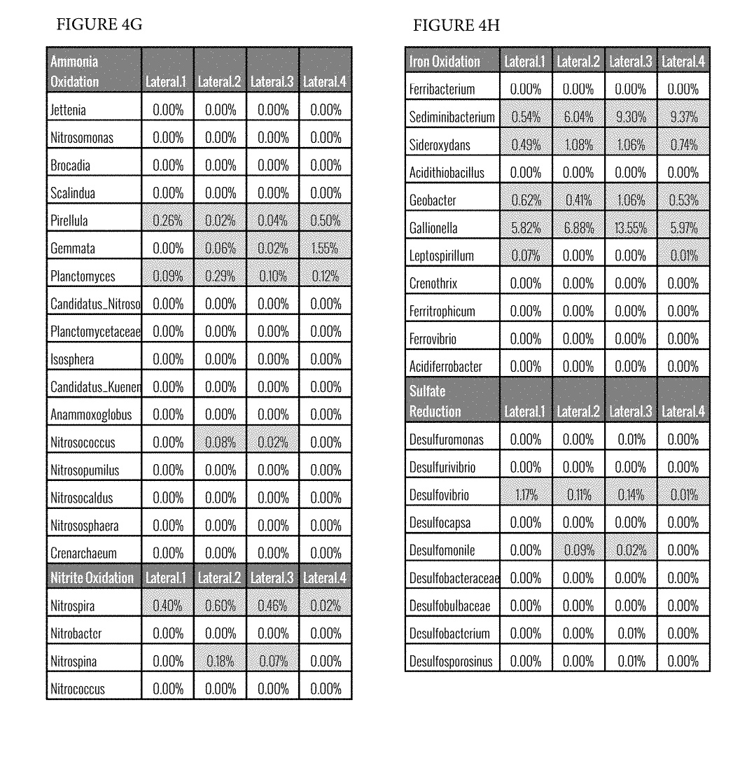

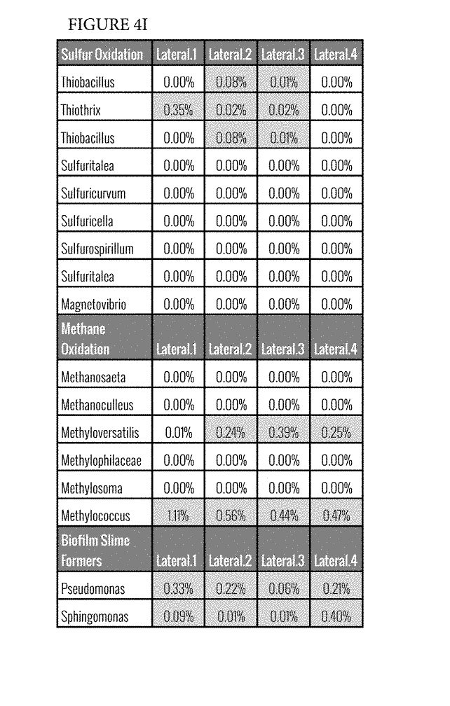

As shown in FIGS. 4A-4I, the DNA sequences can reveal the identity of the microbes found in a sample. In some cases, the relative proportions of each microbe (compared to the whole) can be determined. Further, this information can be aggregated to illuminate the relative proportions of certain types or families of bacteria, or to identify microbes with common functional features.

As further shown in FIG. 4A-4I, families of microbes can be screened to identify microbes in a specific taxonomy (such as cyanobacteria). Such searches can also be used to determine whether certain classes or types of bacteria are absent from a sample.

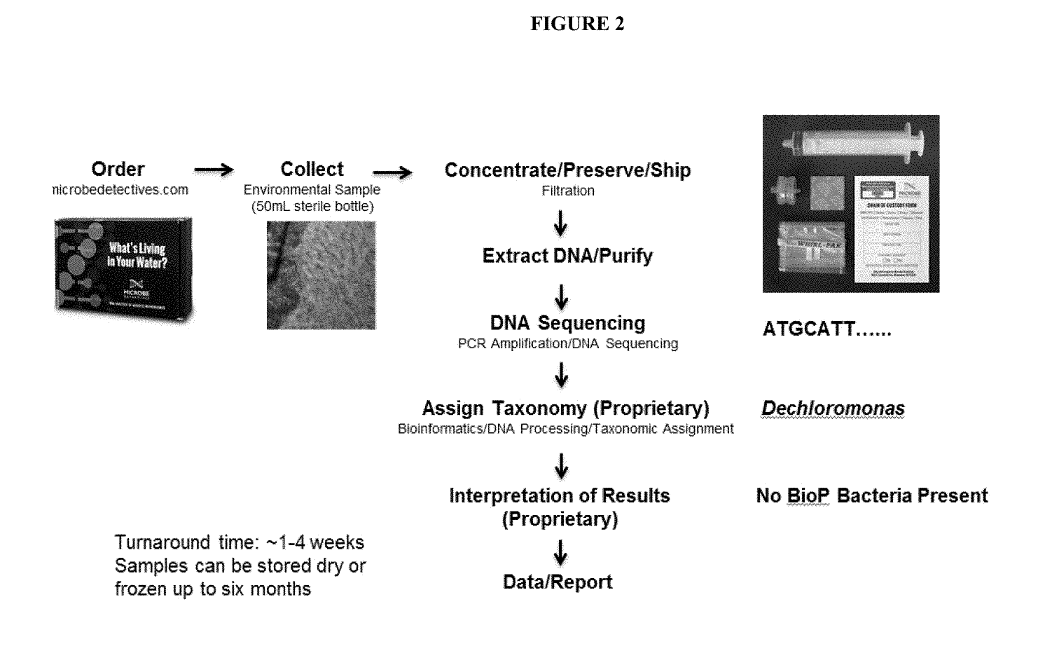

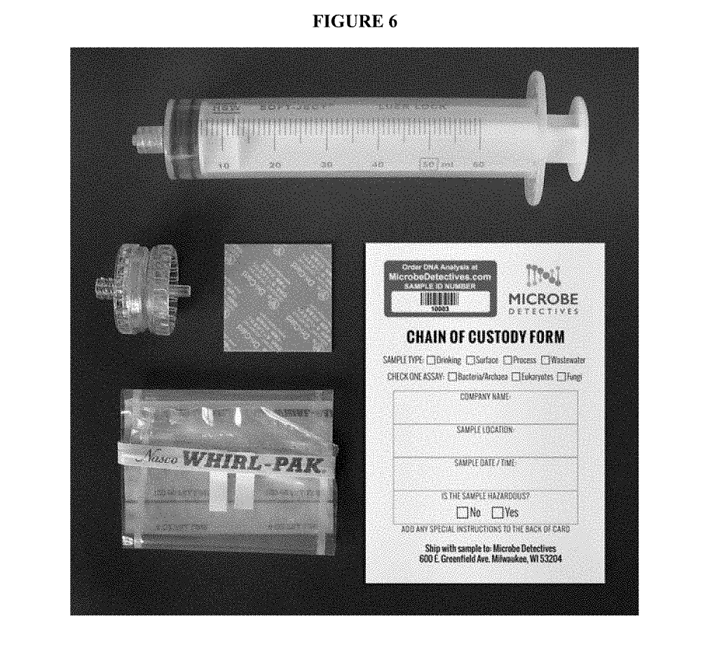

Referring now to FIGS. 2 and 6, an embodiment of the invention can include in one aspect a kit for performing steps in the process and method of the embodiment the invention. Such a kit can include a collector for collecting a liquid sample. For example, the sample can be collected in a container (preferably sterile or sterilized) such as a disposable lidded or capped cup, an Eppendorf tube, a conical centrifuge tube, or other collection container for storing liquid samples. An embodiment of the kit can also include a filter or other membrane or substrate for collecting bacterial or microbial samples. An embodiment of the kit can also include instructions for using the invention. An embodiment of the kit can also include a desiccant card (or other drying material) for keeping collected samples dry during storage or shipping.

As shown in FIGS. 2 and 6, an embodiment of the kit can include a syringe such as, for example, a plastic syringe with a filter unit for attaching to the end of the syringe. In one embodiment the filter unit is preferably sterile and can have a filter housing surrounding the filter, to avoid contamination when handled by the user.

In practicing an embodiment of the invention a user will collect a sample in a clean, preferably sterile, vessel or container, the sample being well-mixed. For example, the syringe of one embodiment can be used to draw water or liquid from the collection vessel into the syringe. An attachable filter unit (such as, for example, a filter protected within a filter housing) can be attached to an end of the syringe of one embodiment, and the sample can be expelled from the syringe and through the filter. In an embodiment the water or liquid can thus be expelled from the syringe, while the microbes remain trapped onto the filter. In one embodiment the filter unit can then be removed. This procedure can be repeated to sample large volumes or liquid.

The filter can be immediately subjected to DNA extraction, amplification, and identification, or can be dried or stored or shipped prior to those steps.

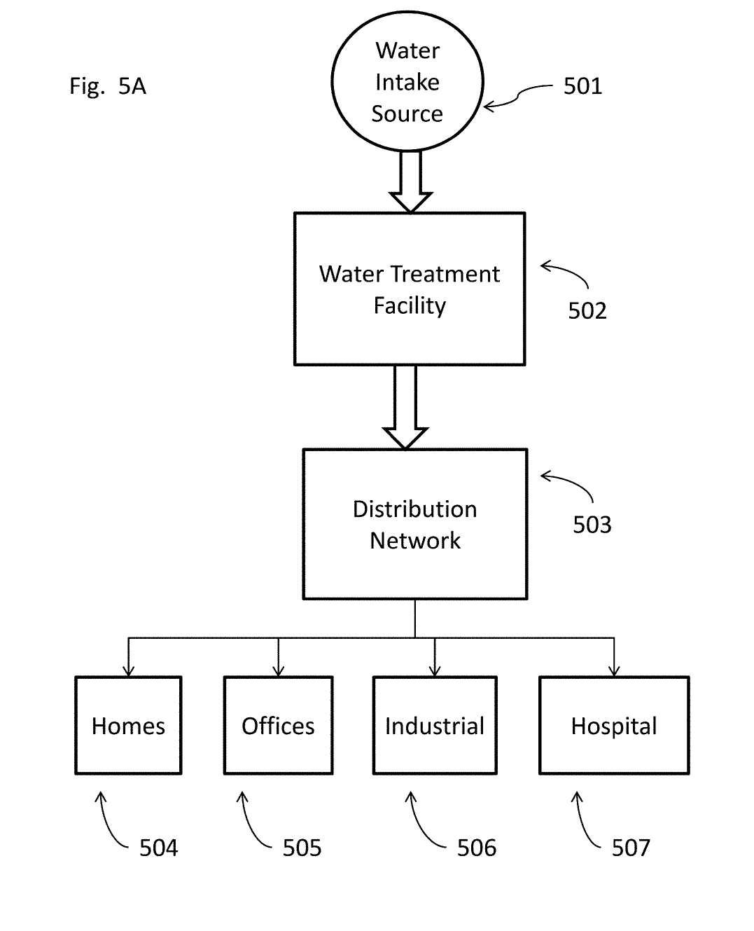

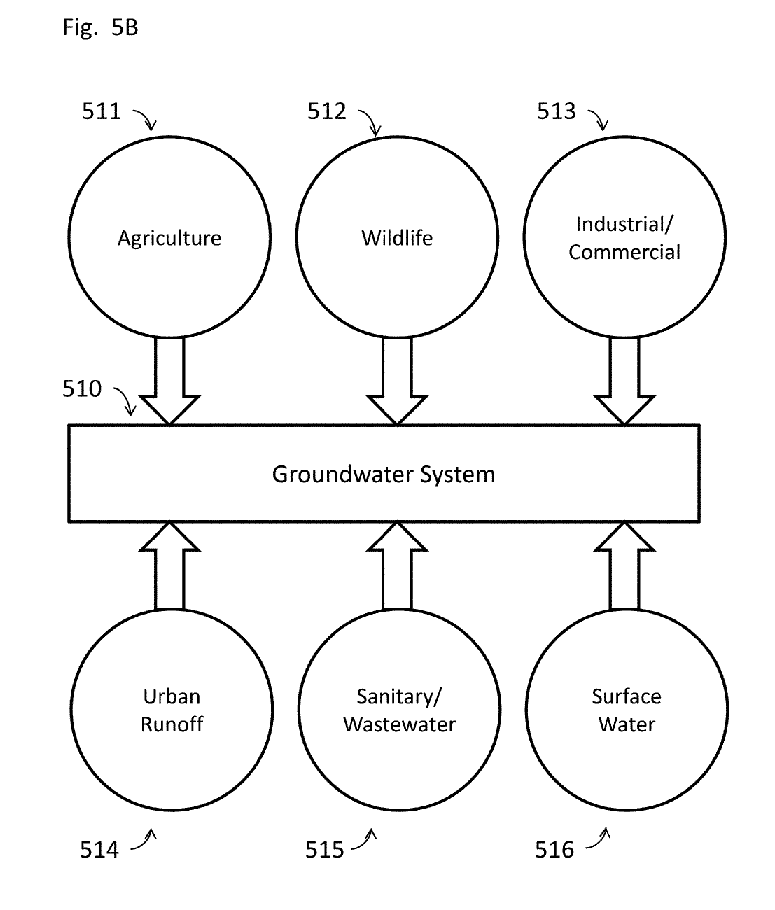

Referring now to FIG. 5A, there is shown a schematic representation of a water distribution system for distribution of, for example, potable drinking water within a community. Raw water is taken from a Water Intake Source (501) such as, for example, a natural source of water such as, for example, a lake, river, or groundwater system. Raw water is then processed at a Water Treatment Facility (502) to meet any applicable water usage standards such as, for example, standards promulgated by the U.S. Environmental Protection Agency for drinkable water or the like. Treated water then can be distributed through a Distribution Network (503). A distribution system may comprise, for example, many miles of pipes and conduits, and may create many possible points of contamination for the treated water. Treated water distributed through the Distribution Network (503) is delivered to water use points such as, for example, a Home (504), an Office (505), an Industrial Facility (506), or a Hospital (507). These water use points may then include further distribution networks of the received treated water to the particular taps or the like at which water is used. It is an object of an embodiment of the invention to be able to use microbial DNA sequencing to test for potential contamination at a variety of test points throughout a water distribution such as, for example, that shown in FIG. 5A to detect, for example, contamination introduced into the treated water after leaving the Water Treatment Facility (502) or even within the water use points such as, for example, a Home (504), an Office (505), an Industrial Facility (506), or a Hospital (507).

Referring now to FIG. 5B, there is shown a diagrammatic representation of potential contamination of groundwater from a plurality of sources. A Groundwater System (510) may include, for example, water held underground in the soil or in pores and crevices in rock, and accessible, for example, through deep wells, aquifers, and the like. A Groundwater System (510) may be subject to microbial contamination from a variety of sources. One example of a potential source of contamination can include, for example, Agriculture (511) in the form of, for example, runoff or drainage that may include animal or plant waste that may seep in to and contaminate the Groundwater System (510). Another example of a potential source of contamination can include, for example, Wildlife (512) in the form of waste products from birds or other animals or decaying plant matter, which may produce waste that can enter and contaminate the Groundwater System (510). Another example of a potential source of contamination can include, for example, Industrial/Commercial (513) or similar human factors which can produce waste or effluent containing microbial contamination which can potentially enter and contaminate a Groundwater System (510). Another example of a potential source of contamination can include, for example, Urban Runoff (514) or similar discharge from human communities, campgrounds, dwellings, and the like which can potentially enter and produce microbial contamination in a Groundwater System (510). Another example of a potential source of contamination can include, for example, Sanitary/Wastewater (515) such as discharge, effluent, or disposal from a wastewater treatment facility or the like which can potentially enter and produce microbial contamination in a Groundwater System (510). Another example of a potential source of contamination can include, for example, Surface Water (516) which may include forms of microbial contamination which may in turn potentially enter and contaminate a Groundwater System (510).

Conventional or previously known methods of detection such contamination as, for example, analysis of a culture taken from the Groundwater System (510) may provide little guidance as to the particular source of contamination, and may screen only for a limited number of different types of microbial contamination. It is an object of an embodiment of the present invention to use microbial DNA sequencing to provide broad screening for a very large variety of potential microbial contaminants. It is a further object of an embodiment of the present invention to use microbial DNA sequencing to identify the particular microbial contaminant, thus providing information as to the potential sources of contamination as an aid in eliminating, curing, limiting, or otherwise providing remediation to contamination detected.

Persons of ordinary skill in the art will appreciate that water systems subject to contamination are known to occur in a variety of forms, both natural and artificial. Natural water systems subject to microbial contamination include not only a Groundwater System (510) as depicted in FIGS. 5A-5B, but other naturally occurring water systems such as oceans, seas, lakes, rivers, streams, wetlands, and their adjacent beaches, banks, waterfronts, and the like. Analogous artificial water systems can include, for example, pools, water parks, spas, and other manmade structures containing water subject to microbial contamination. It is further an object of an embodiment of the invention to use microbial DNA sequencing of samples of such water systems to provide for broad spectrum screening for a very wide variety of potential microbial contaminations beyond the capabilities of commonly used systems today such as, for example, culture growth. It is a further object of an embodiment of the invention to use microbial DNA sequencing to identify specific microbial contaminants in such natural and artificial water systems.

The presence of different specific microbes at different locations is listed. This information can then be used to determine a course of action to identify other sites for subsequent analysis, to determine the likely causes of contamination, and to design a cure and remediation for the contamination problems at this factory.

In an embodiment of the present invention, similar processes and kits can be used to detect contamination in groundwater wells or, for example, lakes or beaches. The process of an embodiment of the present invention can be used to detect unusual contaminant microbes that can indicate, for example, fecal contamination or intrusion of surface water.

The foregoing describe exemplary embodiments of the invention and a person of ordinary skill in the art will recognize that modifications can be made without departing from the spirit and scope of the invention as set forth in the following claims

The present invention is not limited to the particular details of the method/embodiment described, and a person of ordinary skill in the art will understand that other modifications, applications, embodiments, and examples are contemplated within the scope of the present invention. A person of ordinary skill in the art would further recognize that certain other changes can be made in the above-described method without departing from the true spirit and scope of the invention herein involved. For example, the present method can be utilized with other types of water delivery systems, such as water fountains, closed buildings, pools, irrigation systems, waste treatment systems, and more, which transport liquids in a variety of forms and manners. It is intended, therefore, that the subject matter in the above depiction shall be interpreted as illustrative and not in a limiting sense.

What is claimed is:

1. A method of identifying a microbe in a water system comprising:
collecting a microbe from a water sample from a water system;
extracting a microbial DNA sequence from the microbe, the microbial DNA sequence having a hypervariable region unique to the microbe;
amplifying a portion of the hypervariable region located in the microbial DNA sequence; and
determining the sequence of the amplified portion and thereby determining the identity of the microbe from which the amplified portion was obtained.

2. A method according to claim 1 wherein the microbe from the water sample from the water system are in the water sample drawn from the water system.

3. The method according to claim 1 wherein the microbe from the water sample from the water system is deposited in a filter through which the water sample from the water system has been filtered.

4. The method of claim 3, wherein the microbe is collected on a 0.22 μm filter.

5. The method of claim 1, wherein the step of determining the identity of the microbe further comprises accessing a database listing microbial DNA sequences and identifying in the database the hypervariable region located in the microbial DNA sequence.

6. The method of claim 1, wherein the hypervariable region is the fourth hypervariable region in the 16S rRNA gene.

7. The method of claim 6, wherein the DNA sequence is amplified using a first primer and a second primer that amplify the v4 region.

8. The method of claim 7, wherein the first primer comprises sequence SEQ ID:1 and the second primer comprises sequence SEQ ID: 2.

9. The method of claim 7, wherein the DNA sequence is amplified via polymerase chain reaction.

10. The method of claim 1 wherein the water sample from the water system is collected using a kit, the kit comprising:
a sterile container for obtaining the liquid sample;
a sterile filter for collecting the microbe; and
a set of instructions for collecting the microbe.

11. The method of claim 1 where the water system is a naturally occurring body of water.

12. The method of claim 1 where in the water system is a man-made water distribution system.

13. A method of identifying a microbe in a liquid system comprising:
collecting a microbe from a liquid sample from a liquid system;
extracting a microbial DNA sequence from the microbe, the microbial DNA sequence having a hypervariable region unique to the microbe;
amplifying a portion of the hypervariable region located in the microbial DNA sequence; and
determining the sequence of the amplified portion and thereby determining the identity of microbe from which the amplified portion was obtained.

14. The method of claim 13 wherein the liquid is water.

15. The method of claim 13, wherein the hypervariable region is the fourth hypervariable region in the 16S rRNA gene.

16. The method of claim 13, wherein the hypervariable portion of the extracted DNA sequence is amplified by PCR using a first primer having the sequence of SEQ ID:1 and a second primer having the sequence of SEQ ID: 2.


SEQUENCE LISTING (2 sequences)

SEQ ID NO: 1 - DNA, 19 bp, Artificial Sequence
  Sequence found in microbes
gtgycagcmgccgcggtaa

SEQ ID NO: 2 - DNA, 20 bp, Artificial Sequence
  Sequence found in microbes
  misc_feature (8)..(8): n is a, c, g, or t
ggactacnvgggtwtctaat